(19) United States
(12) Patent Application Publication
(10) Pub. No.: US 2017/0164903 A1
(43) Pub. Date: Jun. 15, 2017

(54) AUTONOMOUS BIOELECTRIC PHYSIOLOGICAL SIGNAL ACQUISITION DEVICE

(71) Applicant: RYTHM, Paris (FR)

(72) Inventors: Quentin SOULET DE BRUGIERE, PYLA SUR MER (FR); Hugo MERCIER, CHILLY MAZARIN (FR); Kaveh MOHAMADABADI, PARIS (FR)

(21) Appl. No.: 15/373,971

(22) Filed: Dec. 9, 2016

(30) Foreign Application Priority Data
Dec. 9, 2015 (EP) 15306962.0

(51) Int. Cl. A61B 5/00; G06F 19/00; A61M 21/00

(52) U.S. Cl. CPC A61B 5/7225; A61B 5/4806; A61B 5/6814; A61M 21/00; G06F 19/3406; A61M 2021/0027

(57) ABSTRACT

A method for bioelectric physiological signal acquisition includes:

/a/ providing an autonomous bioelectric physiological signal acquisition device including an electrode and an acquisition chain associated to the electrode,

/b/ acquiring an analog signal representative of a bioelectric physiological signal with the electrode,

/c/ amplifying the analog signal by an amplification factor via a programmable analog amplifier of the acquisition chain,

/d/ converting the amplified analog signal in a digital signal via an analog digital converter of the acquisition chain,

/e/ processing the digital signal to determine whether the digital signal is considered saturated, if the digital signal is considered saturated,

/f/ selecting, in a list of predefined amplification factors, an amplification factor lower than the amplification factor currently applied to the digital signal by the programmable analog amplifier, and

/g/ updating the amplification factor of the programmable analog amplifier to the selected amplification factor.

### FIELD OF THE INVENTION

The instant invention relates to methods for bioelectric physiological signal acquisition and autonomous bioelectric physiological signal acquisition devices.

### BACKGROUND OF THE INVENTION

Bioelectric physiological signals generated by electrical currents within the neurons or the nerves of an individual can be detected by performing electrocardiogram or electroencephalogram for instance. Such measurements have led to breakthrough in the field of biological sciences and present huge potentials in many daily-life fields such as self-quantization, self-optimization or human-computer interfaces to name only a few.

Recently, bioelectric physiological signal acquisition devices have started to be developed with the aim of providing conformable and easy to use way of acquiring bioelectric physiological signal.

Documents WO 2015/017563, CN 103961093, CN 102512159 and WO 2014062738 describe examples of such devices.

These devices usually comprise a few electrodes to acquire an analog signal, filters and converters to obtain a digital signal and a microprocessor to process the signals and send wirelessly said signals to an external server. These devices are designed to be worn by the user during a certain period, for example a period of sleep in the case of sleep tracking devices. These devices may also act on the brain function of the user during this period, for example by emitting sound pattern to stimulate the brain of the user during sleep.

Since these devices may be worn over long periods of time, up to several hours, there is an incentive for the devices to be autonomous and able to process signals without having to communicate with external servers. This way, it is possible to minimize exposure of the user to electromagnetic fields.

However, the computational power and memory required to process the bioelectric signals imposes stringent demands on the power supply and the autonomy of the devices.

There is thus a need to further improve the autonomy of the bioelectric physiological signals acquisition while keeping a high quality and reliability of signals acquisition.

The instant invention has notably for object to improve this situation.

### SUMMARY OF THE INVENTION

To this aim, a first object of the invention is a method for bioelectric physiological signal acquisition comprising:

/a/ providing an autonomous bioelectric physiological signal acquisition device comprising at least one electrode and at least one acquisition chain associated to said electrode,

/b/ acquiring at least one analog signal representative of a bioelectric physiological signal by means of said electrode,

/c/ amplifying said analog signal by an amplification factor by means of a programmable analog amplifier of said acquisition chain,

/d/ converting said amplified analog signal in a digital signal by means of an analog digital converter of said acquisition chain,

/e/ processing said digital signal to determine whether said digital signal is considered saturated,

if said digital signal is considered saturated,

/f/ selecting, in a list of predefined amplification factors, an amplification factor lower than the amplification factor currently applied to the digital signal by the programmable analog amplifier, and

/g/ updating the amplification factor of the programmable analog amplifier to the selected amplification factor.

In some embodiments, one might also use one or more of the following features:

steps /b/ to /g/ are reiterated continuously during a period of acquisition of the device;

if the amplification factor currently applied to the digital signal by the programmable analog amplifier is equal to the lowest amplification factor in the list of predefined amplification factors and the digital signal is considered saturated, the analog digital converter and/or the analog amplifier of the acquisition chain are powered out and steps /d/ to /g/ are not performed anymore;

if an analog digital converter and/or an analog amplifier of an acquisition chain has been powered out, the following steps are periodically performed:

/h/ powering said analog digital converter and/or analog amplifier of an acquisition chain,

/i/ performing at least one iteration of steps /b/ to /e/,

if the digital signal obtained from said at least one iteration is not considered saturated,

/j/ setting back on said analog digital converter and/or analog amplifier for further iterations of steps /b/ to

/e/ until said digital signal is considered saturated, if the digital signal obtained from said at least one iteration is considered saturated,

/k/ powering out said analog digital converter and/or analog amplifier for further iterations of steps /b/ to /g/until step /h/ is performed again;

if the amplification factor currently applied to the digital signal by the programmable analog amplifier is not equal to the highest amplification factor in the list of predefined amplification factors, the following steps are periodically performed:

/l/ selecting, in the list of predefined amplification factors, an amplification factor higher than the amplification factor currently applied to the digital signal by the programmable analog amplifier,

/m/ updating the amplification factor of the programmable analog amplifier to the selected amplification factor, and

/n/ performing at least one iteration of steps /b/ to /g/;

to determine whether a digital signal is considered saturated,

a portion of the digital signal is stored in a buffer memory, preferably a portion with a duration of at least one second,

a ratio of saturated values to non-saturated values in the stored digital signal is computed,

said ratio is compared to a predefined threshold, and

if said ratio is over said predefined threshold, the digital signal is considered saturated;

if the digital signal is considered saturated an amplification level change indicator is written in a memory of the device together with an associated time stamp, and preferably if the analog digital converter of the acquisition chain is powered out a channel power out indicator is further written in said memory together with an associated time stamp;

the provided autonomous bioelectric physiological signal acquisition device comprises a plurality of electrodes and a plurality of respective acquisition chains respectively associated to said electrodes,

said plurality of electrodes acquires a plurality of respective analog signals representative of a plurality of respective bioelectric physiological signals,

said plurality of analog signals are amplified by a plurality of amplification factor by means of a respective plurality of programmable analog amplifier of said plurality of acquisition chains,

said plurality of amplified analog signals are converted in a respective plurality of digital signals by means of a respective plurality of analog digital converters of said plurality of acquisition chains,

said digital signals are independently processed to determine whether each digital signal of the plurality of digital signals is considered saturated,

and for each digital signal of the plurality of digital signals that is considered saturated,

an amplification factor, lower than the amplification factor currently applied to said digital signal by the programmable analog amplifier associated to said digital signal, is selected in a list of predefined amplification factors, and

the amplification factor of the programmable analog amplifier associated to said digital signal is updated to the selected amplification factor.

Another object of the invention is an autonomous bioelectric physiological signal acquisition device comprising

at least one electrode able to acquire an analog signal representative of a bioelectric physiological signal,

at least one acquisition chain associated to said electrode and comprising:

a programmable analog amplifier able to amplify an analog signal by an amplification factor, and

an analog digital converter able to convert an amplified analog signal in a digital signal,

a microcontroller and a memory able to

process a digital signal to determine whether said digital signal is considered saturated, and

if said digital signal is considered saturated,

select, in a list of predefined amplification factors, an amplification factor lower than the amplification factor currently applied to the digital signal by the programmable analog amplifier, and

update the amplification factor of the programmable analog amplifier to the selected amplification factor.

In some embodiments, one might also use one or more of the following features:

the device comprises a plurality of electrodes and a plurality of respective acquisition chains respectively associated to said electrodes;

the acquisition chains are integrated in a single monolithic chip incorporating programmable analog amplifiers and analog digital converters;

the analog digital converter is of the 24 bit delta-sigma (Δ-Σ) type with an output data rate between 250 SPS and 32 kSPS;

the device comprises at least one battery powering at least the acquisition chain, the microcontroller and the memory;

the device further comprises a microprocessor able to receive the digital signal from the microcontroller and to process said digital signal.

With these features, among other advantages, the autonomy of the bioelectric physiological signals acquisition is improved and a high quality and reliability of signals acquisition is provided.

### BRIEF DESCRIPTION OF THE DRAWINGS

Other characteristics and advantages of the invention will readily appear from the following description of several of its embodiments, provided as non-limitative examples, and of the accompanying drawings.

On the drawings:

FIG. 1 is a schematic view in perspective of an autonomous bioelectric physiological signal acquisition device according to an embodiment of the invention,

FIG. 2 is a detailed schematic view of the device of FIG. 1 seen from the top, showing the control module and the batteries of the device,

FIG. 3 is a detailed view of the control module of the device of FIG. 1, showing the printed circuit board and their components.

On the different Figures, the same reference signs designate like or similar elements.

### DETAILED DESCRIPTION OF THE INVENTION

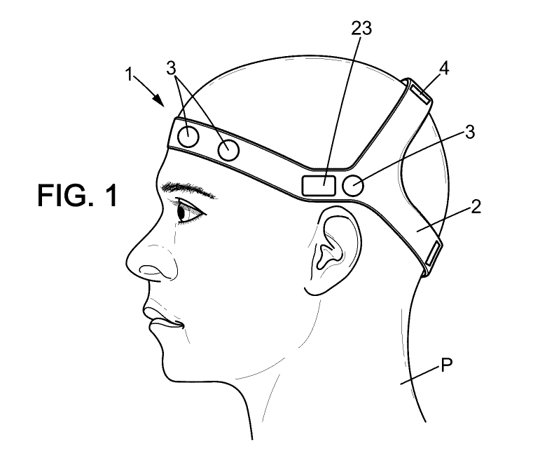

FIG. 1 illustrates an autonomous bioelectric physiological signal acquisition device 1 according to an embodiment of the invention.

The device 1 is adapted to be worn by an individual, for example during a sleep period of the individual.

The device 1 is for example adapted to be worn on the head of the individual.

To this end, the device 1 can comprise one or more support members 2 adapted to at least partially surround the head of the individual so as to be held therein. In the embodiments of FIG. 1, the supports members 2 comprise three branches arranged so as to surround the head of the individual and maintain the device 1.

Alternatively, the device 1 may also be divided into elements, adapted to be worn on various parts of the body of the individual, for example on the head or wrist or on the chest.

The device 1 of FIG. 1 comprises five electrodes 3 able to be in contact with the skin of the individual and to acquire an analog signal. The analog signal can be representative of a bioelectric physiological signal.

Electrodes 3 may be reusable electrodes or disposable electrodes. Advantageously, electrodes 3 are reusable electrodes so as to simplify the daily use of the device. Electrodes 3 may be, in particular, dry electrodes. The electrodes 2 may also be of reusable or disposable textile or silicone electrodes.

In one embodiment of the invention, the device 1 comprises five electrodes 3 arranged, for example on the surface of the scalp of the person P.

The five electrodes 3 may comprise for instance two reference electrodes located on the mastoids, two measurement electrodes located on the forehead and a bias or virtual ground electrode located on the forehead. Such an arrangement is only described as a manner of example and should not be understood as being limitative with respect to the invention.

The five electrodes 3 are held by the supports members 2.

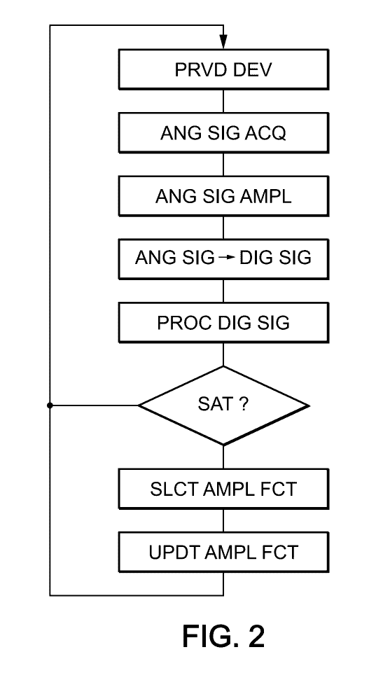

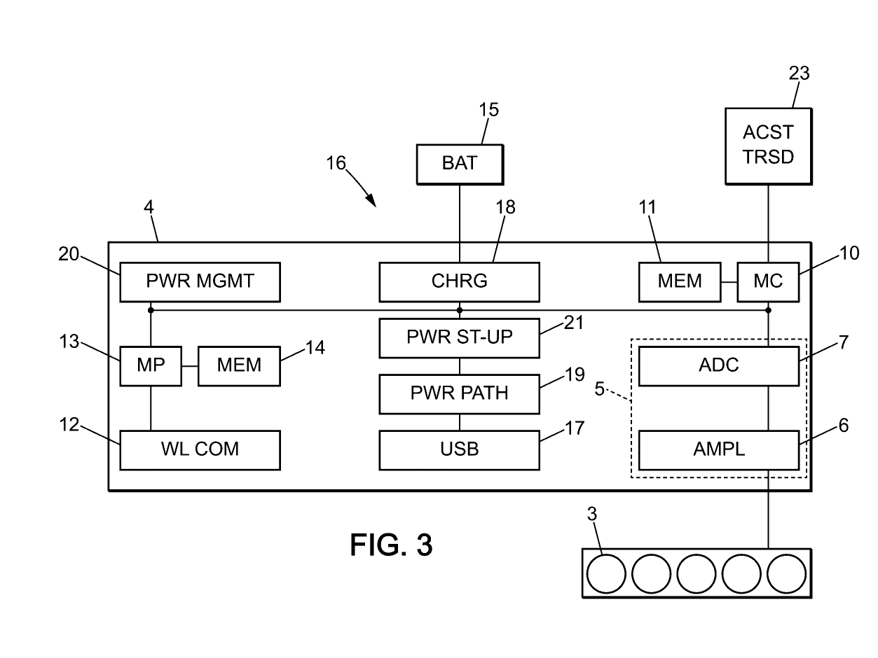

As illustrated on FIGS. 2 and 3, the device 1 further comprises a control module 4 to process and store the signals of the electrodes.

The control module 4 comprises several acquisition chains 5, each acquisition chain 5 being associated to two electrodes 3 of the plurality of electrodes, for instance four acquisition chains 5. Each acquisition chain may advantageously receive signals from two electrodes 3, among the plurality of electrodes 5, in order to perform a differential signal measurement.

The acquisition chains 5 may be integrated in a single monolithic chip 5.

Each acquisition chain 5 comprises at least a programmable differential analog amplifier 6 and an analog digital converter 7.

The programmable differential analog amplifier 6 is able to amplify an analog signal by an amplification factor.

Each acquisition chain 5 may further comprise analog signal filters to filter the analog signal before said signal is amplified by the programmable analog amplifier 6.

The analog signal filters may comprise, for instance, low-pass and/or high-pass filters.

The input referred noise of the analog amplifiers 6 may be around 4 uV at 150 Hz bandwidth. The programmable analog amplifiers 6 may thus have a high input impedance, high common mode rejection ratio, low bias current and low input referred noise to reduce the mentioned noise sources.

The analog amplifiers 6 are programmable gain amplifiers with an adjustable gain, for instance adjustable to at least a 12 times factor of amplification.

The analog digital converters 7 are able to convert an amplified analog signal into a digital signal.

The analog digital converters may have at least 4 channels and comprises 24 bit delta-sigma (Δ-Σ) type ADC with an output data rate between 250 SPS and 32 kSPS.

The four programmable analog amplifiers 6 and the four analog digital converters 7 of the four acquisition chains 5 may form a single monolithic chip 5.

One example of thus a monolithic chip 5 is an analog front-end module with reference ADS1294 of Texas Instrument®.

The control module 4 may further comprise a microcontroller 10 and a memory 11.

The microcontroller 10 is able to continuously receive the digital signal from the acquisition chains 5.

The memory 11 may be constituted of two modules: an internal memory located inside the chip of the microcontroller 10 and an external memory located outside the microcontroller 10, advantageously in proximity with the microcontroller 10. The external memory may have a larger capacity than the internal memory.

The microcontroller 10 is able to continuously store the digital signal in the memory 11.

The microcontroller 10 may be a low power microcontroller. One example of low power microcontroller is a Cortex®-M0+ of ARM®.

The microcontroller 10 and the memory 11 are further able to process a digital signal to determine whether said digital signal is considered saturated.

To determine whether the digital signal is considered saturated, the microcontroller 10 may be able to store a portion of the digital signal in a buffer memory of the memory 11.

Said portion of the digital signal may preferably have a duration of at least one second, for instance 5 seconds.

The microcontroller 10 may then be able to compute a ratio of saturated values to non-saturated values in the stored digital signal.

The microcontroller 10 then compares said ratio to a predefined threshold.

If said ratio is over said predefined threshold, the digital signal is considered saturated.

If said digital signal is considered saturated, the microcontroller 10 may then select, in a list of predefined amplification factors, an amplification factor lower than the amplification factor currently applied to the digital signal by the programmable analog amplifier.

The list of predefined amplification factors may be stored in the memory 11, in particular on the internal memory of the microcontroller 10.

Said selection in the list of amplification factor may, in practice, gives an index value referencing the amplification factor in the list of predefined amplification factors, for instance an index value between 1 and a max value, for instance 7.

The microcontroller 10 can then update the amplification factor of the programmable analog amplifier to the selected amplification factor.

To this aim, the microcontroller 10 may for instance transmit said index value to the programmable analog amplifier.

This way, it is possible to dynamically adjust the amplification level to the quality of the electrode skin contact and to avoid saturated and non-usable signals while still having the highest possible signal levels.

The device 1 may also comprise a microprocessor 13.

The microprocessor 13 is able to communicate with the microcontroller 10 in order to receive the digital signal stored in the memory 11.

The microprocessor 13 is also able to process said digital signal, for example to perform pattern recognition of brain states on said digital signals, for instance pattern recognition of sleep phases.

The microprocessor 13 may be able to achieve 2.5 DMIPS/MHZ (with frequency at 1 GHz). The microprocessor 13 may be for instance a Cortex A9 Series Processor of ARM®.

The microprocessor 13 can then store the signal in a second memory 14 of the device 1.

The second memory 14 may have a larger size than the temporary memory 11. The second memory 14 may comprise a double data rate (DDR) memory and an embedded multi-media controller (eMMC).

The device 1 may also comprise a wireless communication module 12. The microprocessor 13 may be able to control the wireless communication module 12. The wireless communication module 12 may comprises a RF transceiver chip 12 able to transmit and receive data using the Wi-Fi and the Bluetooth standards.

Moreover, the device 1 comprises at least one battery 15. The battery 15 powers the elements of the device 1, in particular at least the acquisition chains 5, the microcontroller 10 and the microprocessor 13, possibly through a power management chip 20.

The device may also comprise a power module 16 with a USB connector 17 and at least one battery charge chip 18. The battery charge chip 18 is able to receive energy from the USB connector 17 and charges said at least one battery 15.

In some embodiments, the device 1 may comprise two batteries 15 or more and the device 1 may then comprise two battery charge chips 18 respectively connected to each battery and may comprise a power-path manager chip 19. The two batteries 15 may then be both connected to the power-path manager chip 19 through the respective battery charge chips 18.

The device 1 may further comprise at least one acoustic transducer 23 able to generate an acoustic stimulation to stimulate the individual.

A method for bioelectric physiological signal acquisition according to an embodiment of the invention can thus comprise the following general steps:

/a/ a step of providing an autonomous bioelectric physiological signal acquisition device,

/b/ a step of acquiring at least one analog signal as described in the present description,

/c/ a step of amplifying said analog signal as described in the present description,

/d/ a step of converting said amplified analog signal into a digital signal as described in the present description, and

/e/ a step of processing said digital signal to determine whether said digital signal is considered saturated as described in the present description.

If said digital signal is considered saturated, the following additional steps may be performed:

/f/ a step of selecting an amplification factor as described in the present description, and

/g/ a step of updating the amplification factor of the programmable analog amplifier to the selected amplification factor as described in the present description.

In some embodiment of the invention, if the digital signal is considered saturated, steps /b/ to /f/ may be reiterated until the digital signal is not considered saturated.

The amplitude of the analog signals from the electrodes is function of the quality of the skin-electrode contact. In particular, the impedance of the skin-electrode contact can vary strongly with changes in the skin-electrode contact.

Thus, if an electrode moves during the acquisition period, the contact can be deteriorated.

The deterioration of the skin-electrode contact can be slight and still allow for a measurement, in which case only the amplification factor needs to be updated, for instance lowered, to prevent saturation of the analog digital converter.

Over, in some cases the deterioration of the skin-electrode contact can be too strong and prevent further measurements of signal.

In this case, the amplification factor currently applied to the digital signal by the programmable analog amplifier may be equal to the lowest amplification factor in the list of predefined amplification factors while the digital signal is still be considered saturated.

The microcontroller 10 may then choose to power out the analog digital converter and/or the analog amplifier of the acquisition chain.

In this embodiment, at least steps /d/ to /g/ of the method according to the invention may then not be performed anymore.

This way, it is possible to take advantage of the temporary lowest quality of acquisition to save energy.

Moreover, if the digital signal is considered saturated, an amplification level change indicator may be written in the memory 11 of the device 1 by the microcontroller 10, together with an associated time stamp.

If the analog digital converter 7 of the acquisition chain 5 is powered out a channel power out indicator may be further written in said memory 11 by the microcontroller 10, together with an associated time stamp.

It may also be possible to periodically test if the quality of the electrode-skin contact has become better.

Thus, in one embodiment, if an analog digital converter and/or an analog amplifier of an acquisition chain has been powered out, the following steps may be periodically performed:

/h/ powering said analog digital converter and/or analog amplifier of an acquisition chain, and

/i/ performing at least one iteration of steps /b/ to /e/.

If the digital signal obtained from said at least one iteration is not considered saturated,

/j/ said analog digital converter and/or analog amplifier may be set back on for further iterations of steps /b/ to /e/, until said digital signal is considered saturated.

If the digital signal obtained from said at least one iteration is still considered saturated,

/k/ said analog digital converter and/or analog amplifier may then be powered out for further iterations of steps /b/ to /e/ until steps /h/ to /j/ are to be performed again.

Similarly, the level of the signal may be tested from times to times, for instance by trying a higher amplification factor for a limited time.

In one example, if the amplification factor currently applied to the digital signal by the programmable analog amplifier is not equal to the highest amplification factor in the list of predefined amplification factors, the following steps may thus be periodically performed:

/l/ selecting, in the list of predefined amplification factors, an amplification factor higher than the amplification factor currently applied to the digital signal by the programmable analog amplifier,

/m/ updating the amplification factor of the programmable analog amplifier to the selected amplification factor, and

/n/ performing at least one iteration of steps /b/ to /g/.

This way, if the digital signal is considered saturated, steps /f/ and /g/ are performed and bring back the amplification factor to its previous level. If the digital signal is not considered saturated, the increase in amplification factor will automatically be kept for further iteration of steps /b/ to /g/.

This way, it is possible to dynamically adjust the amplification level to the quality of the electrode skin contact.

The period of performing steps /l/ to /n/ and the period of performing steps /h/ to /k/ can vary and can in particular be adapted to the current power level of the battery.

In one embodiment of the invention, the microprocessor 13 can be periodically awakened from a sleep state to process the signal stored in the temporary memory 11.

Since the microprocessor 13 has significantly more power consumption compared to the acquisition chain 5 and the microcontroller 10, the microprocessor 13 may only be used in case of need or periodically.

The microprocessor 13 may thus for instance be periodically awakened from a sleep state to receive the digital signal stored in the memory 11 and process said digital signal stored in the memory 11.

The microprocessor 13 may also communicate with the microcontroller 10 to receive the amplification level change indicator(s) and/or the channel power out indicator(s) written in the memory 11.

Moreover, if the microprocessor 13 detects that the signal recorded in the temporary memory 11 is representative of a stimulation-ready state of the individual, the microprocessor 13 may then be continuously kept in an awaken state and command the acoustic transducer 23 to generate acoustic stimulations to the individual.

1-14. (canceled)

15. A method for bioelectric physiological signal acquisition comprising:
/a/ providing an autonomous bioelectric physiological signal acquisition device comprising at least one electrode and at least one acquisition chain associated to said at least one electrode,
/b/ acquiring at least one analog signal representative of a bioelectric physiological signal by means of said at least one electrode,
/c/ amplifying said analog signal by an amplification factor by means of a programmable analog amplifier (6) of said at least one acquisition chain,
/d/ converting said amplified analog signal in a digital signal by means of an analog digital converter of said at least one acquisition chain,
/e/ processing said digital signal to determine whether said digital signal is considered saturated,
if said digital signal is considered saturated,
/f/ selecting, in a list of predefined amplification factors, an amplification factor lower than the amplification factor currently applied to the digital signal by the programmable analog amplifier, and
/g/ updating the amplification factor of the programmable analog amplifier to the selected amplification factor.

16. The method of claim 15, wherein, steps /b/ to /g/ are reiterated continuously during a period of acquisition of the device.

17. The method of claim 15, wherein if the amplification factor currently applied to the digital signal by the programmable analog amplifier is equal to the lowest amplification factor in the list of predefined amplification factors and the digital signal is considered saturated, the analog digital converter and/or the programmable analog amplifier of the acquisition chain are powered out and steps /d/ to /g/ are not performed anymore.

18. The method of claim 17, wherein if an analog digital converter and/or a programmable analog amplifier of an acquisition chain has been powered out, the following steps are periodically performed:
/h/ powering said analog digital converter and/or programmable analog amplifier,
/i/ performing at least one iteration of steps /b/ to /e/,
if the digital signal obtained from said at least one iteration is not considered saturated,
/j/ setting back on said analog digital converter and/or programmable analog amplifier for further iterations of steps /b/ to /e/ until said digital signal is considered saturated,
or if the digital signal obtained from said at least one iteration is considered saturated,
/k/ powering out said analog digital converter and/or programmable analog amplifier for further iterations of steps /b/ to /g/ until step /h/ is performed again.

19. The method of claim 15, wherein, if the amplification factor currently applied to the digital signal by the programmable analog amplifier is not equal to the highest amplification factor in the list of predefined amplification factors, the following steps are periodically performed:
/l/ selecting, in the list of predefined amplification factors, an amplification factor higher than the amplification factor currently applied to the digital signal by the programmable analog amplifier,
/m/ updating the amplification factor of the programmable analog amplifier to the selected amplification factor, and
/n/ performing at least one iteration of steps /b/ to /g/.

20. The method of claim 15, wherein to determine whether a digital signal is considered saturated,
a portion of the digital signal is stored in a buffer memory, preferably a portion with a duration of at least one second,
a ratio of saturated values to non-saturated values in the stored digital signal is computed,
said ratio is compared to a predefined threshold, and
if said ratio is over said predefined threshold, the digital signal is considered saturated.

21. The method of claim 15, wherein if the digital signal is considered saturated an amplification level change indicator is written in a memory of the device together with an associated time stamp, and preferably, if the analog digital converter of the acquisition chain is powered out a channel power out indicator is further written in said memory together with an associated time stamp.

22. The method of claim 15, wherein the provided autonomous bioelectric physiological signal acquisition device comprises a plurality of electrodes and a plurality of respective acquisition chains respectively associated to said electrodes,
wherein said plurality of electrodes acquires a plurality of respective analog signals representative of a plurality of respective bioelectric physiological signals,
wherein said plurality of analog signals are amplified by a plurality of amplification factor by means of a respective plurality of programmable analog amplifier of said plurality of acquisition chains,
wherein said plurality of amplified analog signals are converted in a respective plurality of digital signals by means of a respective plurality of analog digital converters of said plurality of acquisition chains,
wherein said digital signals are independently processed to determine whether each digital signal of the plurality of digital signals is considered saturated,
and wherein for each digital signal of the plurality of digital signals that is considered saturated,
an amplification factor, lower than the amplification factor currently applied to said digital signal by the programmable analog amplifier associated to said digital signal, is selected in a list of predefined amplification factors, and
the amplification factor of the programmable analog amplifier associated to said digital signal is updated to the selected amplification factor.

23. An autonomous bioelectric physiological signal acquisition device comprising
at least one electrode able to acquire an analog signal representative of a bioelectric physiological signal,
at least one acquisition chain associated to said at least one electrode and comprising:
a programmable analog amplifier able to amplify an analog signal by an amplification factor, and
an analog digital converter able to convert an amplified analog signal in a digital signal,
a microcontroller and a memory able to
process a digital signal to determine whether said digital signal is considered saturated, and
if said digital signal is considered saturated,
select, in a list of predefined amplification factors, an amplification factor lower than the amplification factor currently applied to the digital signal by the programmable analog amplifier, and
update the amplification factor of the programmable analog amplifier to the selected amplification factor.

24. The device according to claim 23, the device comprising a plurality of electrodes and a plurality of respective acquisition chains respectively associated to said electrodes.

25. The device according to claim 23, wherein the acquisition chains are integrated in a single monolithic chip incorporating programmable analog amplifiers and analog digital converters.

26. The device according to claim 23, wherein the analog digital converters are of the 24 bit delta-sigma type with an output data rate between 250 SPS and 32 kSPS.

27. The device according to claim 23, wherein the device comprise at least one battery powering at least the at least one acquisition chain, the microcontroller and the memory.

28. The device according to claim 23, further comprising a microprocessor able to receive the digital signal from the microcontroller and to process said digital signal.
